(19) United States
(12) Patent Application Publication
(10) Pub. No.: US 2011/0251189 A1
(43) Pub. Date: Oct. 13, 2011

(54) HYPOXIA TARGETED COMPOUNDS FOR CANCER DIAGNOSIS AND THERAPY

(76) Inventors: Bhaskar Chandra Das, West Nyack, NY (US); Sridhar Mani, Riverdale, NY (US)

(21) Appl. No.: 12/998,703

(22) PCT Filed: Nov. 18, 2009
(86) PCT No.: PCT/US2009/006170

§ 371 Date: Jul. 1, 2011

Related U.S. Application Data
(60) Provisional application No. 61/201,353, filed on Dec. 10, 2008.

(51) Int. Cl. A61K 31/536; C07D 265/36; A61P 35/00; C12N 5/09

(52) U.S. Cl. 514/230.5; 435/375; 544/105

(57) ABSTRACT

The present invention generally relates to oxazine derivative compounds and related compositions and methods for inducing hypoxic tumor cell death, treating cancer and locating a hypoxic tumor in a subject.

### CROSS-REFERENCE TO RELATED APPLICATION

This application claims priority of U.S. Provisional Patent Application No. 61/201,353, filed on Dec. 10, 2008, the content of which is incorporated by reference.

### FIELD OF THE INVENTION

The present invention generally relates to oxazine derivative compounds and related compositions and methods for inducing hypoxic tumor cell death, treating cancer and locating a hypoxic tumor in a subject.

### BACKGROUND OF THE INVENTION

Throughout this application various publications are referred to by Arabic numerals in superscript. Full citations for these references may be found at the end of the specification immediately preceding the claims. The disclosures of these publications are hereby incorporated by reference in their entireties into the subject application to more fully describe the art to which the subject application pertains.

Hypoxia is a universal hallmark of tumor cells in vivo. Within the tumor microenvironment, it contributes towards resistance to radiation and chemotherapy.^{1,2} The inability to treat hypoxic tumor cells effectively represents opportunities for providing novel compounds and strategies for this unmet medical need.³

Hypoxia activated prodrugs has been the archetypical model upon which newer analogs have been made. In this model, several drugs have entered the clinical testing phase. The most advanced is tirapazamine, which is in phase III clinical testing in a variety of solid tumor malignancies. However, tirapazamine lacks the ability for good tumor penetration, and has low in vivo potency at doses that are non-toxic (or have tolerable side-effects) to humans.⁴ Another promising hypoxia-activated prodrug, banoxantrone (AQ4N)^{5,6,7} has been previously investigated. However, there remains some clinical issues with AQ4N, like mucosal discoloration. There, however, has been clinical activity documented in lymphomas. Other drugs belonging to this class of hypoxia agents include 2-[-(2-bromoethyl)-2,4-dinitro-6-[[[2-phosphonooxyl]ethyl]amino]carbonyl]aniline]-ethyl methanosulfonate (PR-1044).⁸

The hypoxia-activated phosphoramidate DNA cross-linking mustards (e.g., dinitrobenzamide mustards) represent another class of hypoxia activated DNA damaging therapeutics. The most successful in this class are 5-nitrothiophene- and 5-nitrofuran-triggered prodrugs of phosphoramidate toxins. The prototypes in this class are cyclophosphamide and ifosfamide. However, both compounds (FDA approved for cancer treatment) show low hypoxia selectivity and hence, are even cytotoxic to normal (and/or normoxic) cells. Other derivatives include more potent hypoxia-activated achiral phosphoramidates.⁹

There are also other strategies that take advantage of the hypoxic tumor microenvironment. These include use of recombinant anaerobic bacterium (e.g., clostridium like C. novyi-NT) that are able to release enzymes in hypoxic conditions that convert 5-fluorocytosine (5-FC) to 5-fluorouracil (5-FU).¹⁰

There remains a pressing need for potent compounds that specifically target hypoxic cells.

### SUMMARY OF THE INVENTION

The present inventors have discovered a series of small molecules, derived from oxazine, which have selective toxicity against tumor cells under hypoxic conditions.

The present invention is directed to a compound having the formula:

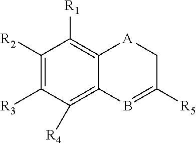

wherein A is CH, CR₁₁, O, SH, S═O, or SO₂; R₁, R₂, R₃, R₄, and R₁₁ are independently a halogen, hydroxy, NO₂, an aryl, an alkyl, an aralkyl or hydrogen; B is N or CH; and R₅ is hydrogen or

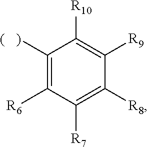

wherein R₆, R₇, R₈, R₉, and R₁₀ are independently a halogen, hydroxyl, an alkyl or hydrogen, and wherein ( ) represents the point of attachment to the main structure.

The present invention is further directed to a compound having the formula:

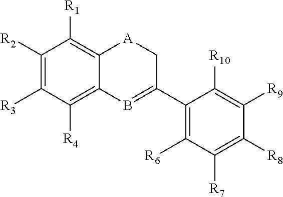

wherein A is CH, CR₁₁, O, SH, S═O, or SO₂; R₁, R₂, R₃, R₄ and R₁₁ are independently a halogen, hydroxy, NO₂, an aryl, an alkyl, an aralkyl or hydrogen; B is N or CH; and R₆, R₇, R₈, R₉, and R₁₀ are independently a halogen, hydroxy, an alkyl or hydrogen.

The present invention is further directed to a method for preparing any of the claimed compounds.

The present invention is further directed to a pharmaceutical composition comprising any of the claimed compounds.

The present invention is further directed to a method for inducing hypoxic tumor cell death comprising contacting the tumor cell with any of the claimed compounds.

The present invention is further directed to a method for treating cancer in a subject comprising administering to the subject a therapeutically effective amount of the claimed compounds.

The present invention is further directed to a method for determining the location of a tumor in a subject comprising (a) administering to the subject any one of the claimed compounds further comprising a detectable marker, and (b) determining the location of the compound in the subject, thereby determining the location of the tumor in the subject.

### BRIEF DESCRIPTION OF THE FIGURES

FIG. 1. RT-Quantitative PCR of Hypoxia Markers.

FIG. 2. Chemiluminescence Assay.

FIG. 3. Survival (MTT) assays of HepG2 cells exposed to compound(s) under normoxic or hypoxic conditions.

FIG. 4A-4B. HepG2 Clonogenic Assays.

FIG. 5. Graphical representation of colony count for compounds 3 and 7.

FIG. 6. Graphical representation of colony count for compoundB47.4.

### DETAILED DESCRIPTION OF THE INVENTION

The present invention provides a compound having the formula:

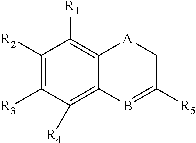

wherein A is CH, CR₁₁, O, SH, S═O, or SO₂; R₁, R₂, R₃, R₄ and R₁₁ are independently a halogen, hydroxy, NO₂, an aryl, an alkyl, an aralkyl or hydrogen; B is N or CH; and R₅ is hydrogen or

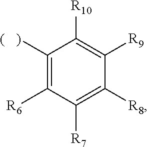

wherein R₆, R₇, R₈, R₉, and R₁₀ are independently a halogen, hydroxyl, an alkyl or hydrogen, and wherein ( ) represents the point of attachment to the main structure.

The present invention further provide a compound having the formula:

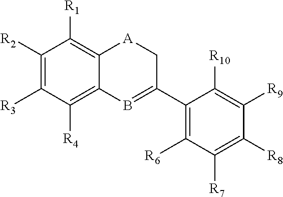

wherein A is CH, CR₁₁, O, SH, S═O, or SO₂; R₁, R₂, R₃, R₄ and R₁₁ are independently a halogen, hydroxy, NO₂, an aryl, an alkyl, an aralkyl or hydrogen; B is N or CH, and R₆, R₇, R₈, R₉, and R₁₀ are independently a halogen, hydroxy, an alkyl or hydrogen.

In another embodiment of the claimed invention, the compound has the formula:

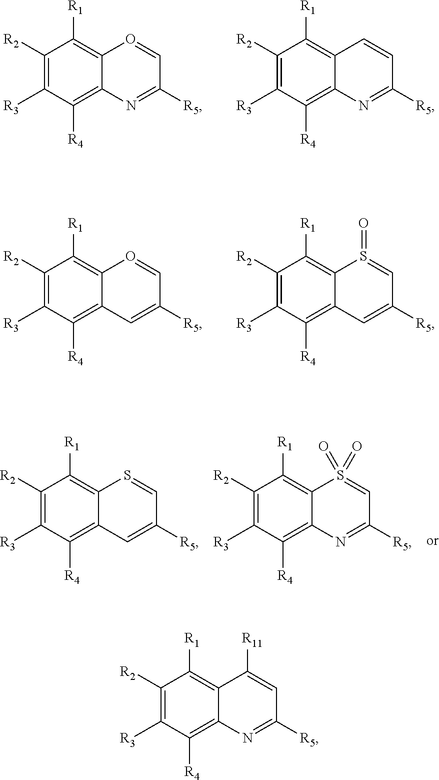

wherein R₁, R₂, R₃, R₄, and R₁₁ are independently a halogen, NO₂, an aryl or hydrogen; and R₅ is hydrogen or

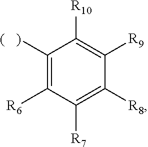

wherein R₆, R₇, R₈, R₉, and R₁₀ are independently a halogen, hydroxy, an alkyl or hydrogen, and wherein ( ) represents the point of attachment to the main structure.

In another embodiment of the claimed invention, the compound has the formula:

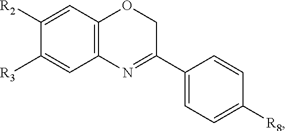

wherein R₂ and R₃ are independently a halogen, NO₂, an aryl, hydroxy, hydrogen, or C₁-C₃ alkyl, and R₈ is a halogen, hydroxyl, hydrogen or C₁-C₃ alkyl.

Specific compounds of the claimed invention include, but are not limited to, compounds having the following formulas:

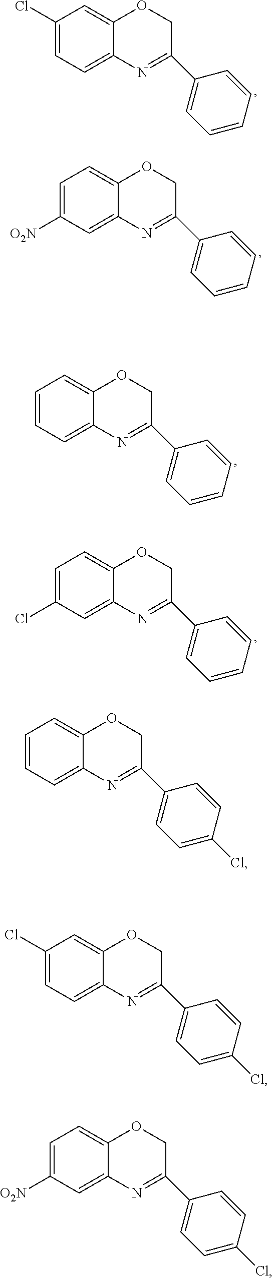

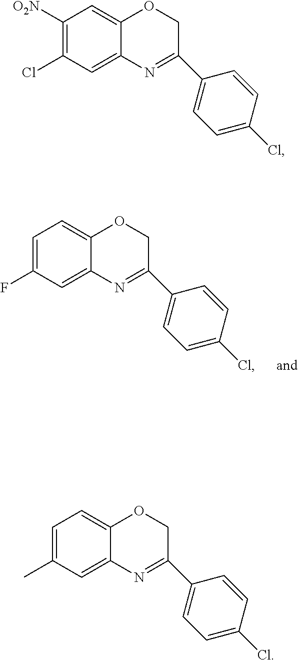

The term “alkyl” is intended to include straight- and branched-chain alkyl groups, as well as cycloalkyl groups. The same terminology applies to the non-aromatic moiety of an aralkyl radical. Examples of alkyl groups include: methyl group, ethyl group, n-propyl group, iso-propyl group, n-butyl group, iso-butyl group, sec-butyl group, t-butyl group, n-pentyl group, 1,1-dimethylpropyl group, 1,2-dimethylpropyl group, 2,2-dimethylpropyl group, 1-ethylpropyl group, 2-ethylpropyl group, n-hexyl group and 1-methyl-2-ethylpropyl group. In one embodiment, the alkyl is preferably methyl, ethyl or C₁-C₃ alkyl. More preferably, the alkyl is methyl or ethyl. Most preferably, the alkyl is methyl.

The term “aralkyl” means an alkyl radical having an aryl substituent.

The term “aryl” means an aromatic radical having 6 to 18 carbon atoms and includes heteroaromatic radicals. Examples include monocyclic groups, as well as fused groups such as bicyclic groups and tricyclic groups. Some examples include phenyl group, indenyl group, 1-naphthyl group, 2-naphthyl group, azulenyl group, heptalenyl group, biphenyl group, indacenyl group, acenaphthyl group, fluorenyl group, phenalenyl group, phenanthrenyl group, anthracenyl group, cyclopentacyclooctenyl group, and benzocyclooctenyl group, pyridyl group, pyrrolyl group, pyridazinyt group, pyrimidinyl group, pyrazinyl group, triazolyl group, tetrazolyl group, benzotriazolyl group, pyrazolyl group, imidazolyl group, benzimidazolyl group, indolyl group, isoindolyl group, indolizinyl group, purinyl group, indazolyl group, furyl group, pyranyl group, benzofuryl group, isobenzofuryl group, thienyl group, thiazolyl group, isothiazolyl group, benzothiazolyl group, oxazolyl group, and isoxazolyl group.

The term “halogen” includes fluorine, chlorine, bromine and iodine.

In another embodiment of the claimed invention, the compound comprises a detectable marker. Numerous detectable markers and methods for detecting compounds labeled with these detectable markers are known in the art, and include, but are not limited to, fluorescent proteins, fluorescent moieties, and radioactive moieties. In one embodiment, the radioactive moiety is a positron emission imaging agent. As used herein, a positron emission imaging agent is any agent that is detectable via positron emission tomography. Examples of a positron emission imaging agent include, but are not limited to, carbon-11, nitrogen-13, oxygen-15, and fluorine-18.

The present invention further provides a pharmaceutical composition comprising any one of the above-described compounds and a pharmaceutically acceptable carrier. As used herein, a “pharmaceutically acceptable” carrier shall mean a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The present invention further provides a method for inducing hypoxic tumor cell death comprising contacting the hypoxic tumor cell with any of the above-described compounds. As used herein, a “hypoxic tumor cell” shall mean a tumor cell located in tissue derived of an adequate supply of oxygen.

The present invention further provides a method for treating cancer in a subject comprising administering to the subject a therapeutically effective amount of any of the above-described compounds. The cancer is preferably present in a solid tumor, a semi-solid tumor or a liquid tumor. The tumor is preferably present in a hypoxic environment. The cancer can be, for example, cervical carcinoma, hepatocellular carcinoma, a lymphoma, Burkitt's lymphoma, nasopharangeal carcinoma, Hodgkin's disease, skin cancer, primary effusion lymphoma, multicentric Castleman's disease, T-cell lymphoma, B-cell lymphoma, splenic lymphoma, Kaposi's sarcoma, post-transplant lymphoma, brain cancer, osteosarcoma, mesothelioma cervical dysplasia, anal cancer, colorectal cancer, cervical cancer, vulvar cancer, vaginal cancer, penile cancer, oropharyneal cancer, nasopharyneal cancer, oral cancer, liver cancer, renal cancer, melanoma, adult T-cell leukemia, or hairy-cell leukemia. As used herein, a “therapeutically effective amount” of a compound for treating cancer shall mean an amount of the compound capable of killing cancer cells, reducing cancer cell metastasis, proliferation or spreading, or alleviating one or more symptoms of cancer in the subject.

The above methods are useful for the treatment of cancer in humans and other animals. Thus, the term “subject” as used herein includes both humans and other animals. Preferably, the subject is a human being.

In the preferred embodiment of the invention, the above-described compounds can easily be administered parenterally such as for example, by intramuscular, intrathecal, subcutaneous, intraperitoneal, intravenous bolus injection or intravenous infusion. Parenteral administration can be accomplished by incorporating the compounds of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, the above-described compounds can be designed for oral, nasal, lingual, sublingual, nasal, buccal and intrabuccal administration and made without undue experimentation by means well known in the art, for example with an inert diluent or with an edible carrier. The compounds may be enclosed in gelatin capsules or compressed into tablets. For the purpose of oral therapeutic administration, the compounds of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

Rectal administration includes administering the compounds into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the pharmaceutical composition through the skin. Transdermal formulations include patches (such as the well-known nicotine patch), ointments, creams, gels, salves and the like.

The present invention further provides a method for determining the location of a hypoxic tumor in a subject comprising (a) administering to the subject any of the above-described compounds comprising a detectable marker, and (b) determining the location of the compound in the subject, thereby determining the location of the hypoxic tumor in the subject. Since the claimed compounds specifically target hypoxic tumor cells, administering to the subject a compound comprising a detectable marker will determine the location of any hypoxic tumors in the subject.

The present invention further provides uses for inducing hypoxic cell death, treating cancer in a subject, manufacturing a pharmaceutical composition for inducing hypoxic cell death in a subject, manufacturing a pharmaceutical composition for treating cancer in a subject, and manufacturing a compound for determining the location of a hypoxic tumor in a subject.

This invention will be better understood from the Experimental Details, which follow. However, one skilled in the art will readily appreciate that the specific methods and results discussed are merely illustrative of the invention as described more fully in the claims that follow thereafter.

### Experimental Details

Hypoxic cells are highly reductive in nature. One approach to the selective delivery of a cytotoxic species utilizes the reductive process to enhance catalysis of a specific activation reaction. Cellular reduction of aromatic nitro compounds is mediated by a range of nitro reductases which convert the nitro group, in a series of electron transfer steps, to the hydroxylamine and ultimately the amine. For one-electron nitroreductases, the first species formed is the nitro radical anion, which in the presence of oxygen is efficiently back-oxidized to the starting nitro compound. The rate of oxidation is dependent on the intracellular concentration of oxygen. Thus the initial step in the pathway gives the reaction its oxygen sensitivity. Subsequent steps give the retention that is necessary to differentiate between normoxic and hypoxic tissues. Such scavenging of the nitro radical anion is suppressed in hypoxic tissue, leading to net reduction of the nitro group. The conversion of free nitro radical anion to nitroso and subsequent free amine is the crucial step to kill hypoxic cells.

Therefore, it was hypothesized that, if another reductive group was introduced to increase the reduction potential of the parent compound, then reduction of the nitro group will favor in the amine step. It was found that dihydro 1,3 oxazine derivatives are potent antitumor agents.¹¹ Since the oxazine moiety shows antitumor activity, it was envisioned that introduction of a nitro group to the 1,4 oxazine moiety shown below would be a good precursor for a novel hypoxia agent.

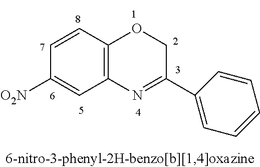

### Materials

(RPMI1640) heat-inactivated fetal bovine serum (FBS), trypsin-EDTA (0.25%), and penicillin-streptomycin were purchased from GIBCO/Invitrogen (Carlsbad, Calif.). Charcoal/dextran treated FBS was purchased from Hyclone (Logan, Utah). HepG2 cells were obtained from ATCC (Manassas, Va.).

### Methods

### Synthesis of 3-phenyl-2H-benzo[b][1,4]oxazine derivatives

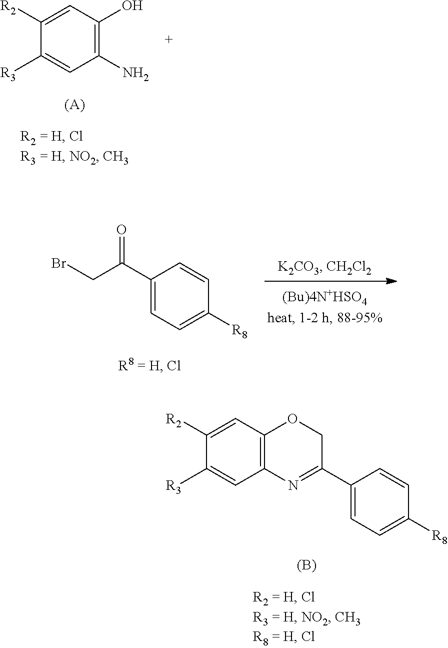

The 3-aryl-2H-benzo[b][1,4]oxazines were synthesized by modifying the existing methods.¹² To a solution of 2-aminophenol (A) (0.001 mol) in dichloromethane (40 mL), aqueous potassium carbonate (20% w/v) and tetrabutylammonium hydrogen sulfate (0.0005 mol) was added and the mixture was stirred for 2 h at room temperature. After 2 h, 2-Bromo-4-chloroacetophenone (0.01 mol) in 20 mL dichloromethane was added drop-wise through a course of 15 minutes and the resultant mixture was refluxed till completion for 4-6 h. The organic layer was extracted with dichloromethane and dried over sodium sulfate evaporated in vacuum to give a crude, solid product. The solid was then re-crystallized with hot ethanol to get pure yield 87-95% (B). New compounds are characterized by 1H, 13C NMR and HRMs and known compounds compared with that of existing analytical data.

Cell Culture and Cytotoxicity Assays. HepG2 cells (passage 2 from frozen stock) were cultured in RPMI 1640 containing 10% FBS. The tests were performed in 96-well plates with stock cells split equally into duplicate plates—one for normoxic and the other for hypoxic conditions. The drug(s) or vehicle (0.1% DMSO) was applied to the cells in log-exponential growth phase under normoxic conditions (5% CO₂; 37 C) for 48 hours or under normoxic conditions (22 hr) followed by 2 hr hypoxic conditions (1% O₂, 5% CO₂, 94% N₂ at 37° C.) then followed by reoxygenation for 24 hrs. For the hypoxia experiments, a glove chamber with two-way pressure value and one-way N₂ inflow was used. Hypoxia conditions were met using methylene blue dye indicator (i.e. 0.015% methylene blue, 6% dextrose, 6 mM NaOH) which correlated with the O₂ pressure in the glove box when measured continuously with a Clark-type electrode.¹³

At 48 hrs, a tetrazolium component (MTT) assay (Promega, Madison, Wis.; Cell Titer 96 Non-Radioactive Cell Proliferation Assay) was performed following an established protocol in the laboratory. Since these assays depend on mitochondrial function, which may be affected by the state of oxygenation, clonogenic assays to verify survival function were also performed. It is important to note that the data presented for the MTT assay normalizes the OD₅₉₅ for the drug treated wells (minus background) to its own vehicle treated well(s) under either normoxic or hypoxic conditions. These values are then plotted as a percentage (×100%) surviving 48 hrs of drug(s) or vehicle treatment under the said condition. These analyses have also been performed previously for other hypoxia studies.^{14,15}

In the HepG2 clonogenic assays, 3.5×10³ per well were seeded in 6-well plates using RPMI1640 and 10% FBS. After 24 hrs of growth at 37° C., the cells were exposed to vehicle or drug(s). After 10 days incubation with drug(s) or control, the wells were washed with PBS twice, then fixed in 10% acetic acid for 10 min, then stained with crystal violet for 10 min, and finally rinsed with distilled water at room temperature. The colonies (>50 μm likely representing >50 cells) were then visually counted in randomly chosen 1-mm×1-mm grid repeated three times. Statistical analyses of the mean number of colonies were performed by using the Mann-Whitney t test, with significant differences established as P<0.05 as previously published by the laboratory.¹⁶ For the hypoxia conditions, after 24 hr growth in normoxic conditions as above, the vehicle or drug(s) treated cells were exposed to hypoxic environment (1% O₂, 5% CO₂, 94% N₂ at 37° C.) for 5 hrs, followed by re-oxygenation (5% CO₂; 37° C.) for the remaining 10 days.

Quantitative RT-PC. Total cellular RNA was extracted using TRIzol reagent (Invitrogen) according to manufacturer's instructions. Reverse transcription was first carried out to generate cDNA using total RNA with random oligodT primers (Invitrogen), dNTPs, and SuperScript III reverse transcriptase (Invitrogen). Real-time polymerase chain reaction (RT-PCR) was performed using the generated cDNA. Quantitative assessment of DNA amplification was carried out via FAM™ Dye and MGB probe using the PRISM 7700 Sequence Detector (Applied Biosystems, Foster City, Calif.) and specific primers for human VEGF(Hs00900054_ml)(amplicon length ˜60 bp)HIF-1(Hs00936372_ml)(˜72 bp), p21 (Hs01121168_ml)(˜72 bp) and β-actin (NM_{—}001101.2; Human ACTB Endogenous Control Probe.(˜171 Primers were synthesized by IDT Technologies (Coralville, Iowa).

The following cycling parameters were used for the PCR: 50° C. for 2 min, 95° C. for 10 min, followed by 40 cycles of 95° C. for 15 s and 60° C. for 1 min. A final heating step up to 95° C. was performed to obtain melting curves of the final PCR products. The fluorescence threshold cycle value (Cₜ) was obtained for each curve and normalized to that obtained for the GAPDH housekeeping gene in the same sample to normalize for discrepancies in sample loading. The differences in Cₜ values between treated and control samples was then computed and exponentially multiplied to the base of 2 to obtain relative differences in expression levels. All experiments were carried out in duplicates and independently performed at least three times.

Chemiluminescence. MSD's chemiluminescence detection technology was used to determine fold VEGF (Human VEGF Mab Clone#26503; R&D Systems, Minneapolis, Minn.) in hypoxic versus normoxic HepG2 cells in accordance with manufacturer instructions (Meso Scale Discovery; Gaithersburg, Md.). In this assay, cell lysate (μg) is coated on 96-well MULTI-ARRAY® MSD high bind plates. The wells are incubated at room temperature for 2 hr, and then blocked with MSD Blocker A solution diluted in PBS for 1 hr at room temperature. After addition of primary antibody (1 μg/ml) for 1 hr at room temperature, the wells are washed with Blocker A solution and secondary antibody (1 μg/ml). After an additional 1 hr of incubation, the wells are washed and refilled with 150 μL MSD Read Buffer (1×) with surfactant. Experiments were performed in duplicate and repeated twice.

To analyze VEGF secretion, HepG2 cells were seeded in 12-well plates, cultured to 50% confluence, and switched to fresh media conditioned at normoxia or hypoxia for 5 hr. Twenty-four hours later the supernatants in wells were collected, cleared by centrifugation and stored at −20° C.

Statistical Analysis. Data were analyzed by student t-test (Prism 4.0a software; GraphPad Software, San Diego, Calif.).

### Results

A library of 3-phenyl-2H-benzo[b][1,4]oxazine derivatives (Table 1) was synthesized and their cytotoxicity activity was tested against hypoxic and normoxic cells.

TABLE 1
Library of 3-phenyl-2H-benzo[b] [1,4] Oxazine Derivatives. 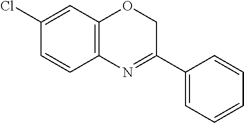
1 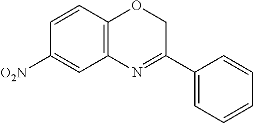
2 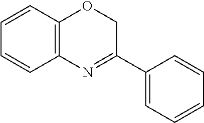
3 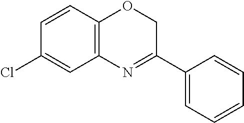
4 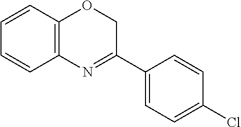
5 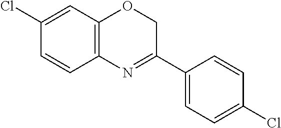
6 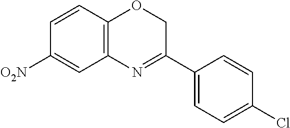
7 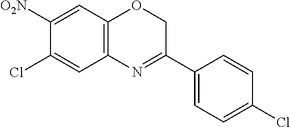
8 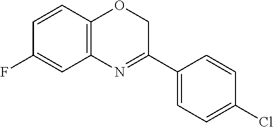
9 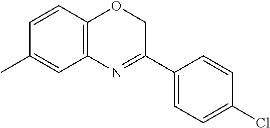
10

The data indicate that the proposed chemistry around oxazine can yield compounds with differential activity in hypoxic versus normoxic conditions. The data indicates that Cpd 3 (which was modified to Cpd 7) can yield additional compounds that have a greater differential activity than the parent molecules.

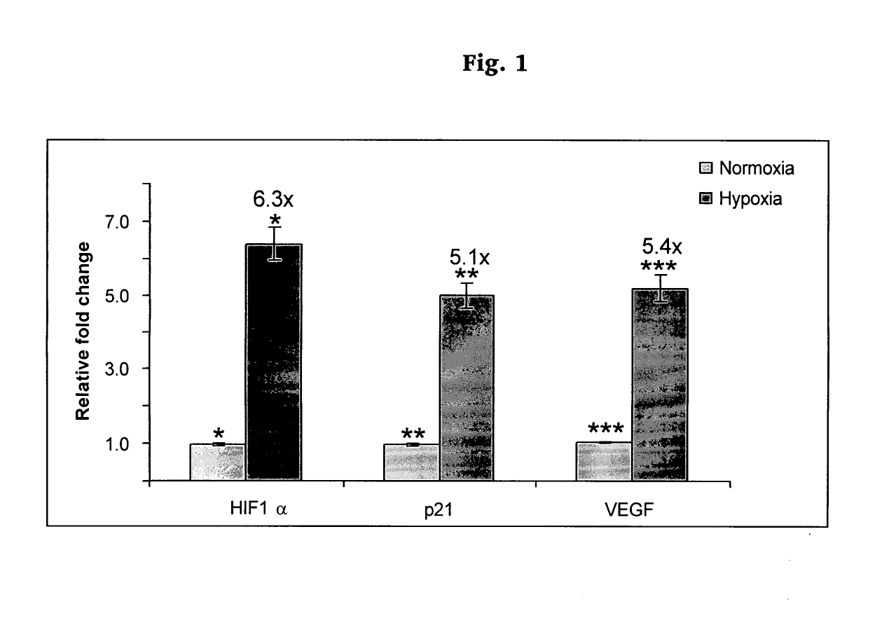

The data indicates that hypoxic HepG2 cells are able to be obtained upon transient exposure to hypoxic conditions (FIG. 1: >6-fold increase in HIF1α, >5-fold increase in p21 and VEGF compared to normoxic conditions). It is noted that although HIF1α protein abundance increases due to increased stability (a major mechanism), there is precedence for other pathways that increase HIF1α that include transcription. Similar findings are true for p21 and VEGF¹⁷⁻¹⁹.

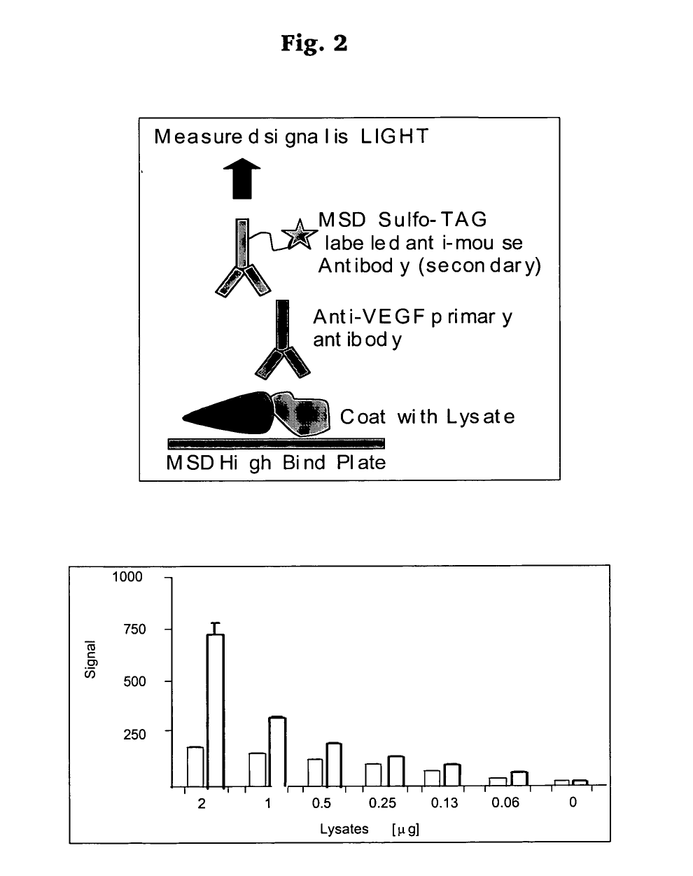

In this setting, Cpd 3 & 7 is significantly more toxic to hypoxic exposure than to normoxic exposure. An increase in VEGF protein in hypoxic cell lysate and media is are also shown, suggesting that VEGF is likely synthesized and excreted (FIG. 2).

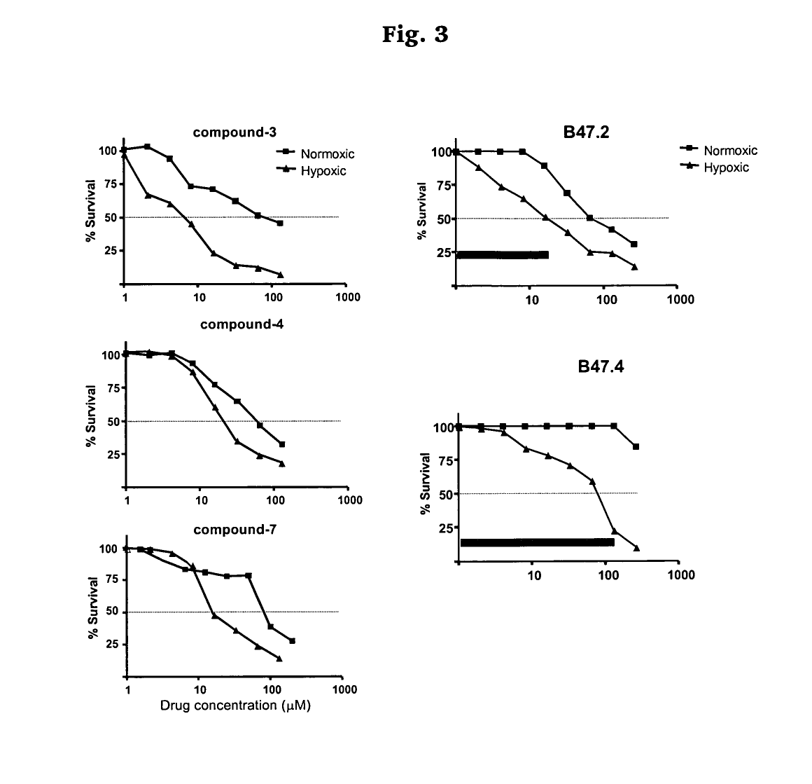

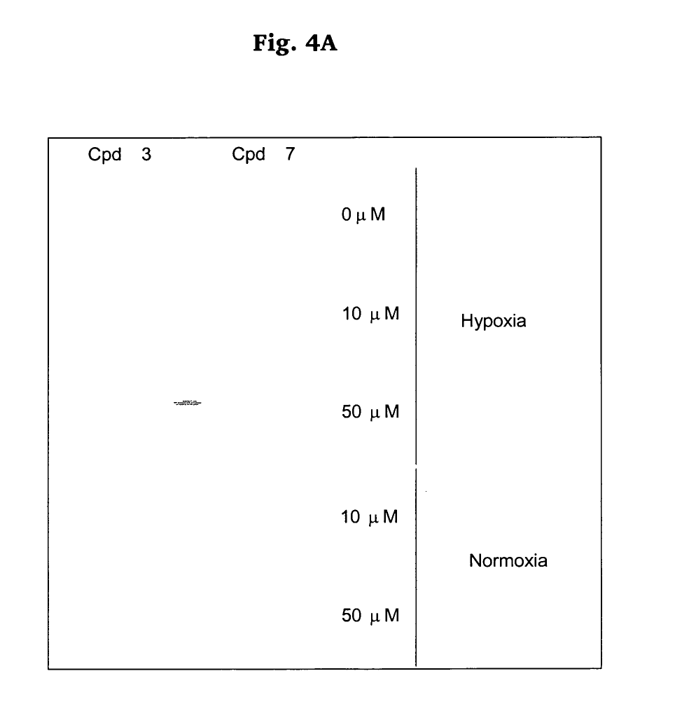

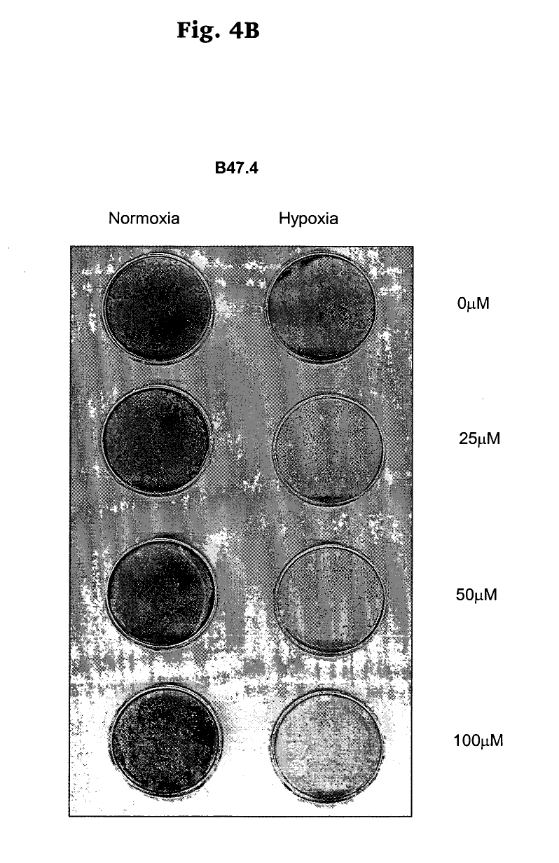

The clonogenic assays (FIGS. 4A and 4B) are fairly accurate and in general correlate with the MTT results (FIG. 3). However, the former seems to be more accurate in depicting cytotoxic exposure as it does not rely on mitochondrial metabolism (which can independently be affected under hypoxic conditions).

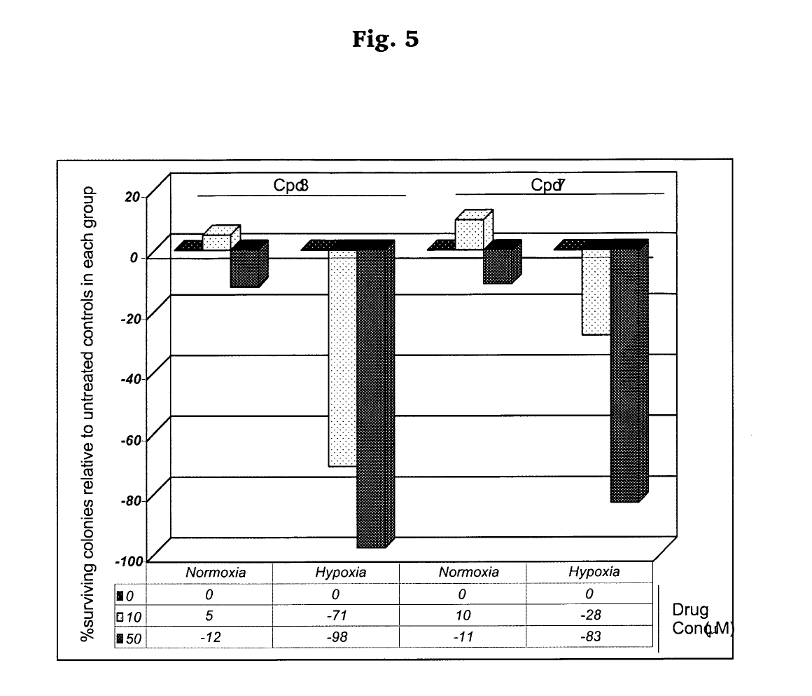

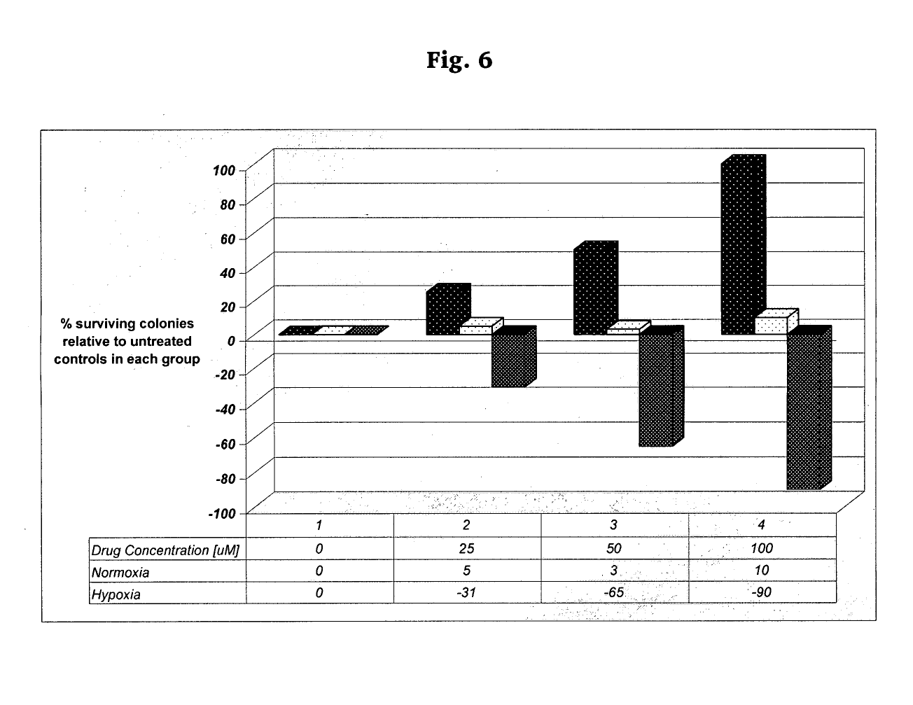

The quantitated colony count from the clonogenic assays show that both Cpds 3 & 7 are minimally toxic to normoxic cells at concentrations ˜10 μM(Cpd3˜105% of colonies relative to controls & Cpd 7˜110% relative to controls are alive) (FIG. 5). In hypoxic conditions, the cytotoxicity is significant in that for Cpd 3 & 7˜71% & 28% of colonies are dead, respectively (p<0.001, comparing this to control treated cells; also see Table 2).

However, at 50 μM concentration of drug(s), there is slight cytotoxicity to normoxic cells (Cpd 3˜12% & Cpd 7˜11% of cells are dead compared with controls). B. In hypoxic conditions, virtually all the cells are dead (Cpd3˜98%; Cpd7˜83% dead; p<0.00001, comparing this to control treated hypoxic conditions).

TABLE 2
Inhibitory Concentration Values (MTT Assay) in HepG2 Cells

| Compound Number | IC₅₀ (normoxia) (μM) | IC₅₀ (hypoxia) (μM) |
|---|---|---|
| 3 | 87 ± 1.3 | 10.765 ± 1.4 |
| 4 | 78 ± 1.2 | 30 ± 2.4 |
| 7 | 78 ± 1.4 | 20 ± 1.2 |
| B47.2 (Compound 6) | 88 ± 2.4 | 28 ± 1.6 |
| B47.4 (Compound 10) | >600 μM | 87 ± 1.8 |

### CONCLUSION

In conclusion, a library of 3-phenyl-2H-benzo[b][1,4]oxazine derivatives was synthesized and their cytotoxicity activity were tested against hypoxic and normoxic cells. 1,4-oxazine analogs were developed for the purposes of bioreductive and oxidative biotransformation in hypoxic cells. In particular, the 3-phenyl-2H-benzo[b][1,4]oxazine moiety substituted at 6 position nitro group plays the dominate role in biological activity. In the context of cancer chemotherapy, this approach led to the discovery of active compounds.

### REFERENCES

1. Dewhirst M W, Cao Y, Moeller B. Cycling hypoxia and free radicals regulate angiogenesis and radiotherapy response. Nat Rev Cancer 2008; 8(6):425-37.
2. Bristow R G, Hill R P. Hypoxia and metabolism. Hypoxia, DNA repair and genetic instability. Nat Rev Cancer 2008; 8(3):180-92.
3. Bache M, Kappler M, Said H M, Staab A, Vordermark D. Detection and specific targeting of hypoxic regions within solid tumors: current preclinical and clinical strategies. Curr Med Chem 2008; 15(4):322-38.
4. Marcu L, Olver I. Tirapazamine: from bench to clinical trials. Curr Clin Pharmacol 2006; 1(1):71-9.
5. Rooney P H T C, McFadyen M C, Melvin W T, Murray G I. The role of cytochrome P450 in cytotoxic bioactivation: future therapeutic directions. Curr Cancer Drug Targets 2004; 4(3):257-65.
6. Albertella M R L P, Jones P H, Phillips R M, Rampling R, Burnet N, Alcock C, Anthoney A, Vjaters E, Dunk C R, Harris P A, Wong A, Lalani A S, Twelves C J. Hypoxia-selective targeting by the bioreductive prodrug AQ4N in patients with solid tumors: results of a phase I study. Clin Cancer Res 2008; 14(4):1096-104.
7. Papadopoulos K P, Goel S, Beeram M, Wong A, Desai K, Haigentz M, Milian M L, Mani S, Tolcher A, Lalani A S, Sarantopoulos J. A Phase 1 Open-Label, Accelerated Dose-Escalation Study of the Hypoxia-Activated Prodrug AQ4N in Patients with Advanced Malignancies. Clin Cancer Res 2008 14(21):7110-5.
8. Patterson A V F D, Edmunds S J, Gu Y, Singleton R S, Patel K, Pullen S M, Hicks K O, Syddall S P, Atwell G J, Yang S, Denny W A, Wilson W R. Mechanism of action and preclinical antitumor activity of the novel hypoxia-activated DNA cross-linking agent PR-104. Clin Cancer Res 2007; 13(13):3922-32.
9. Duan J X J H, Kaizerman J, Stanton T, Evans J W, Lan L, Lorente G, Banica M, Jung D, Wang J, Ma H, Li X, Yang Z, Hoffman R M, Ammons W S, Hart C P, Matteucci M. Potent and highly selective hypoxia-activated achiral phosphoramidate mustards as anticancer drugs. Med Chem 2008; 51(8):2412-20.
10. Dang L H, Bettegowda C, Huso D L, Kinzler K W, Vogelstein B. Combination bacteriolytic therapy for the treatment of experimental tumors. Proc Natl Acad Sci USA. 2001 Dec. 18; 98(26):15155-60; Bettegowda C, Dang L H, Abrams R, Huso D L, Dillehay L, Cheong I, Agrawal N, Borzillary S, McCaffery J M, Watson E L, Lin K S, Bunz F, Baidoo K, Pomper M G, Kinzler K W, Vogelstein B, Zhou S. Overcoming the hypoxic barrier to radiation therapy with anaerobic bacteria. Proc Natl Acad Sci USA. 2003 Dec. 9; 100(25):15083-8; Agrawal N, Bettegowda C, Cheong I, Geschwind J F, Drake C G, Hipkiss E L, Tatsumi M, Dang L H, Diaz L A Jr, Pomper M, Abusedera M, Wahl R L, Kinzler K W, Zhou S, Huso D L, Vogelstein B. Bacteriolytic therapy can generate a potent immune response against experimental tumors. Proc Natl Acad Sci USA. 2004 Oct. 19; 101(42):15172-7; Brown N L, Lemoine N R. Clinical trials with GDEPT: cytosine deaminase and 5-fluorocytosine. Methods Mol Med 2004; 90: 451-7.
11. Kuehne. M. E.; Konopka. E. A. J. Med. Pharm. Chem. 1962, 51, 257-280.
12. Shridhar. D. R.; Reddy. C. V.; Sastry, O. P.; Bansal. O. P.; Rao. P. P. Synthesis, 1981, 912-913.
13. Atkinson H J S L. An oxygen electrode microrespirometer. J Exp Biol 1973; 59:247-53.
14. Wang L, Gao J, Dai W, Lu L. Activation of Polo-like Kinase 3 by Hypoxic Stresses. J Biol Chem 2008; 283(38):25928-35.
15. Unruh A, Ressel, Anke, Mohamed, Hamid G, Johnson, Randall S, Nadrowitz, Roger, Richter, Eckart, Katschinski, Dorthe M, Wenger, Roland H. The hypoxia-inducible factor-1[alpha] is a negative factor for tumor therapy. Oncogene 2003; 22:3213-20.
16. Wu K, Wang C, D'Amico M, et al. Flavopiridol and Trastuzumab Synergistically Inhibit Proliferation of Breast Cancer Cells: Association with Selective Cooperative Inhibition of Cyclin D1-dependent Kinase and Akt Signaling Pathways. Mol Cancer Ther 2002; 1(9):695-706.
17. Belaiba R S, Bonello S, Zahringer C, Schmidt S, Hess J, Kietzmann T, Gorlach A.
Hypoxia up-regulates hypoxia-inducible factor-1alpha transcription by involving phosphatidylinositol 3-kinase and nuclear factor kappaB in pulmonary artery smooth muscle cells. Mol Biol Cell. 2007 December; 18(12):4691-7.
18. Mori T, Ssaki J, Aoyama Y, Sera T. Modulation of endogenous VEGF-A expression under hypoxia by using artificial transcription factors. Nucl Acids Symp Series 2008, 52:187-188.
19. Wang, G. L., Jiang, B.-H., Rue, E. A., and Semenza, G. L. Hypoxia-inducible factor 1 is a basic-helix-loop-helix-PAS heterodimer regulated by cellular O2 tension. 1995 Proc. Natl. Acad. Sci. USA 92, 5510-5514.

1. A compound having the formula: 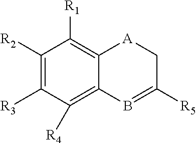
wherein A is CH, CR₁₁, O, SH, S═O, or SO₂;
R₁, R₂, R₃, R₄ and R₁₁ are independently a halogen, hydroxy, NO₂, an aryl, an alkyl, an aralkyl or hydrogen;
B is N or CH; and
R₅ is hydrogen or 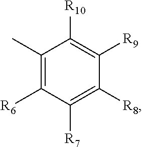
wherein R₆, R₇, R₈, R₉, and R₁₀ are independently a halogen, hydroxyl, an alkyl or hydrogen.

2. The compound of claim 1 having the formula:

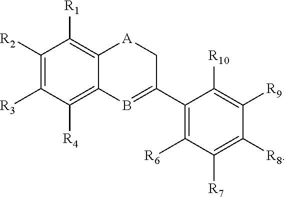

3. The compound of claim 1, wherein the compound has the formula: 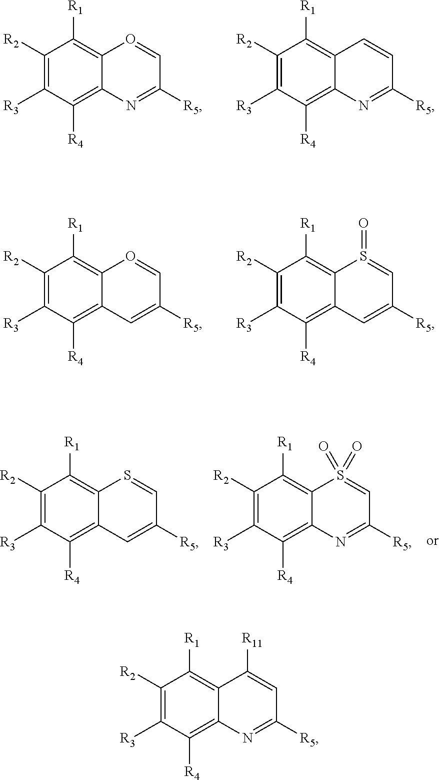
wherein R₁, R₂, R₃, R₄, and R₁₁ are independently a halogen, NO₂, an aryl or hydrogen.

4. The compound of claim 1, wherein the compound has the formula: 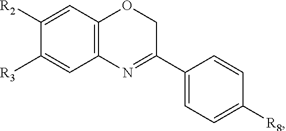
wherein R₂ and R₃ are independently a halogen, NO₂, an aryl, hydroxy, hydrogen, or C₁-C₃ alkyl; and
R₈ is a halogen, hydroxyl, hydrogen or C₁-C₃ alkyl.

5. The compound of claim 1, wherein the compound is:

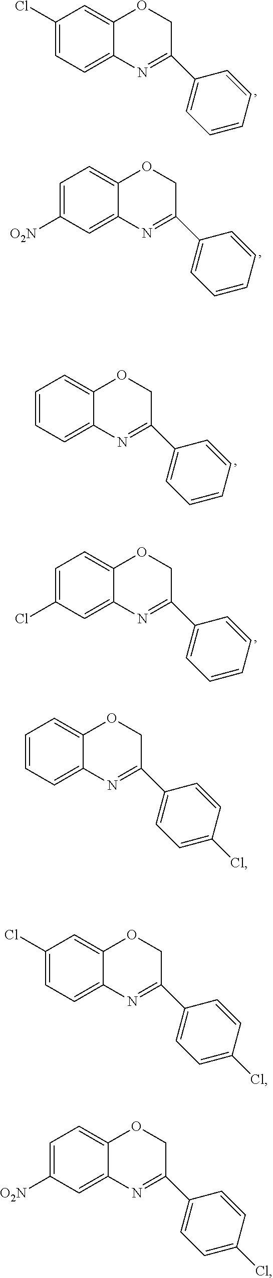

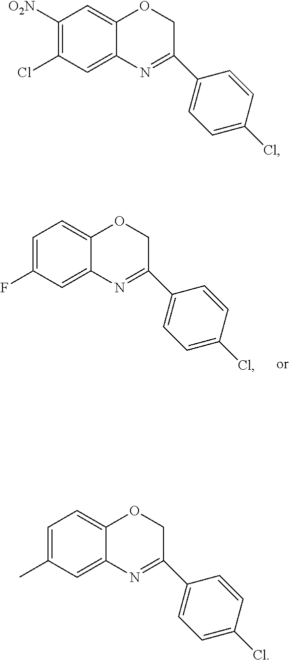

6. The compound of claim 1, wherein the compound comprises a detectable marker.

7. The compound of claim 6, wherein the detectable marker is a fluorescent protein, a fluorescent moiety, or a radioactive moiety.

8. The compound of claim 7, wherein the radioactive moiety is a positron emission imaging agent.

9. The compound of claim 8, wherein the positron emission imaging agent comprises carbon-11, nitrogen-13, oxygen-15, or fluorine-18.

10. A pharmaceutical composition comprising the compound of claim 1, and a pharmaceutically acceptable carrier.

11. A method for inducing hypoxic tumor cell death comprising contacting a tumor cell with the compound of claim 1.

12. A method for treating cancer in a subject comprising administering to the subject a therapeutically effective amount of the compound of claim 1.

13. A method for determining the location of a tumor in a subject comprising:
administering to the subject the compound of claim 1; and
(b) determining the location of the compound in the subject,
thereby determining the location of the tumor in the subject.

14-18. (canceled)
